(19) United States
(12) Patent Application Publication
(10) Pub. No.: US 2006/0148802 A1
(43) Pub. Date: Jul. 6, 2006

(54) Use of compounds that stimulate cgmp

(75) Inventors: Helmut Haning, Wuppertal (DE); Erwin Bischoff, Wuppertal (DE); UIrich Niewohner, Wermelskirchen (DE); Maria Niewohner, Wermelskirchen (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Healthcare AG, Leverkusen (DE)

(21) Appl. No.: 10/516,778

(22) PCT Filed: May 30, 2003
(86) PCT No.: PCT/EP03/05695

§ 371 Date: Sep. 8, 2005

(30) Foreign Application Priority Data
Jun. 3, 2002 (DE) 102 24 462.6

(51) Int. Cl. A61K 31/53

(52) U.S. Cl. 514/246

(57) ABSTRACT

The invention relates to the use of compounds that stimulate cGMP, in particular imidazo[1,3,5]triazinones, for producing a medicament for the treatment and/or prophylaxis of diseases, in which the clinical picture can be improved and/or cured by the improvement of the micro-circulation of tissue containing a phosphodiesterase that metabolises cGMP.

The present invention relates to the use of cGMP-stimulating compounds, in particular of imidazo [1,3,5]-triazinones for producing a pharmaceutical for the treatment and/or prophylaxis of diseases in which an improvement in and/or a cure of a syndrome can be achieved by improving the microcirculation of a tissue which contains a cGMP-metabolizing phosphodiesterase.

Compounds having a cGMP-stimulating effect have been disclosed.

The synthesis of imidazo[1,3,5]triazinones is described in J. Org. Chem. (1979), 44(10), 1740-2; in J. Org. Chem. (1979), 44(22), 3835-9; in J. Org. Chem. (1981), 46(18), 3681-5 and J. Chem. Res. Synop. (1994), (3), 96-7. These publications do not contain any report of a biological effect.

Imidazo[1,3,5]triazinones having an antiviral effect and/or an antitumor effect are described in Nucleosides Nucleotides (1987), 6(4), 663-78; in Eur. J. Med. Chem. (1992), 27(3), 259-66; in J. Heterocycl. Chem. (1993), 30(5), 1341-9; in J. Med. Chem. (1995), 38(18), 3558-68 and Biorg. Med. Chem. Lett. (1996), 6(2), 185-8. Most of the compounds mentioned in these references were prepared as guanine or guanosine analogs and are therefore as a rule substituted by —NH₂, —SH or —H in the 2 position. None of the described compounds contains a phenyl ring or a substituted phenyl ring in the 2 position. None of the described compounds has been reported to have an inhibitory effect against phosphodiesterases.

The compounds which are used in accordance with the invention are potent inhibitors of cyclic guanosine 3′,5′-monophosphate-metabolizing phosphodiesterases (cGMP-PDEs) . In accordance with the nomenclature of Beavo and Reifsnyder (Trends in Pharmacol. Sci. 11, 150-155, 1990), these phosphodiesterases are the phosphodiesterase isoenzymes PDE-I, PDE-II and PDE-V.

WO 0147928 describes imidazo[1,3,5]triazinones which are suitable, inter alia, for treating erectile dysfunction and impotence.

An increase in the concentration of cGMP can lead to curative, antiaggregatory, antithrombotic, antiproliferative, antivasospastic, vasodilatory, natriuretic and diuretic effects. It can have an effect on the short-term or long-term modulation of vascular and cardiac inotropy, on cardiac rhythm and on stimulus conduction in the heart (J. C. Stoclet, T. Keravis, N. Komas and C. Lugnier, Exp. Opin. Invest. Drugs (1995), 4(11), 1081-1100).

The relaxing effect on the smooth musculature leads to a curative improvement in the microcirculation in tissues which contain cGMP-metabolizing phosphodiesterases.

The present invention relates to the use of cGMP-stimulating compounds, in particular of imidazo[1,3,5]-triazinones of the general formula (I) 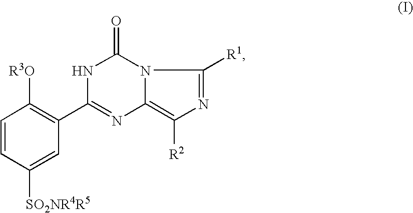
in which
R¹ is straight-chain or branched alkyl having up to 4 carbon atoms,
R² is straight-chain or branched alkyl having-up to 4 carbon atoms or is (C₃-C₈)-cycloalkyl,
R³ is hydrogen or straight-chain or branched alkyl having up to 4 carbon atoms,
R⁴ and R⁵ are identical or different and are hydrogen, (C₁-C₆)-alkoxy or hydroxyl or are (C₁-C₈)-alkyl which is optionally substituted, up to 3 times, identically or differently, by hydroxyl, (C₁-C₆)-alkoxy or radicals of the formulae 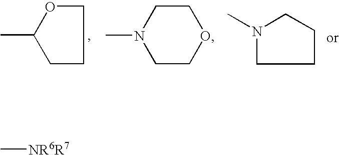
   in which
   R⁶ and R⁷ are identical or different and are hydrogen or (C₁-C₆)-alkyl,
   and/or, for its part, (C₁-C₈)-alkyl is optionally substituted by phenyl or phenoxy which, for their part, are optionally substituted, once to three times, identically or differently, by halogen, hydroxyl, (C₁-C₆)-alkoxy, (C₁-C₆)-alkyl or a radical of the formula —SO₂NR⁸R⁹₁
   in which
   R⁸ and R⁹ are identical or different and are hydrogen or (C₁-C₆)-alkyl,
      or
R⁴ is hydrogen or methyl
   and
R⁵ is radicals of the formulae 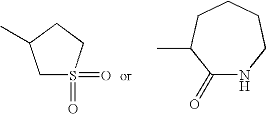
   or
   is phenyl which is optionally substituted, up to 3 times, identically or differently, by halogen, acetyl, (C₁-C₆)-alkoxy or radicals of the formulae 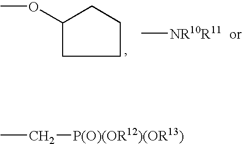
   in which
   R¹⁰ and R¹¹ are identical or different and are hydrogen or (C₁-C₄)-alkyl,
   R¹² and R¹³ are identical or different and are hydrogen or (C₁-C₆)-alkyl,
      or
R⁴ and R⁵, together with the nitrogen atom to which they are bonded, are radicals of the formulae 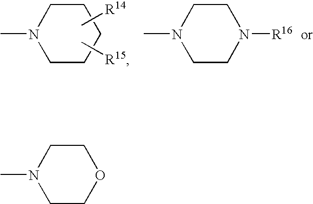
   in which
   R¹⁴ and R¹⁵ are identical or different and are hydroxyl, hydrogen or (C₁-C₄)-alkyl which is optionally substituted by hydroxyl,
   or
   R¹⁴ is hydrogen and
   R¹⁵ is a radical of the formula 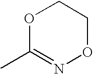
   or
   R¹⁴ and R¹⁵ together form a radical of the formula ═N—O—CH₃,
   R¹⁶ is hydrogen or (C₁-C₆)-alkyl which is optionally substituted by hydroxyl, or
      is a 5- to 6-membered, aromatic heterocycle having up to 3 hetero atoms from the series, S, N and/or O,
         and the salts, N-oxides, hydrates and hydrates of the salts and also isomeric forms thereof, for producing pharmaceuticals for the treatment and/or prophylaxis of coronary heart disease, cardiac insufficiency, pulmonary hypertension, bladder diseases, prostate hyperplasia, nitrate-induced tolerance and diseases of the eye such as glaucoma, for the treatment or prophylaxis of central retinal or posterior cilliary arterial occlusion, central retinal venous occlusion, optical neuropathy such as anterior ischemic optical neuropathy and glaucomatous optical neuropathy, and also macular degeneration and diabetes, in particular diabetic gastroparesis, for the treatment of disturbances in the peristalsis of the stomach and esophagus, of female infertility, premature labor, preeclampsia, alopecia, psoriasis, the renal syndrome, cystic fibrosis and cancer, for improving perception, for improving concentration performance, for improving learning performance and/or memory performance, in particular if the disturbance is a consequence of dementia, for improving perception, concentration performance, learning performance and/or memory performance following cognitive disturbances, as occur, in particular, in connection with situations/diseases/syndromes such as mild cognitive impairment, age-associated learning and memory disturbances, age-associated memory loss, vascular dementia, craniocerebral trauma, stroke, dementia which occurs after strokes (post-stroke dementia), post-traumatic craniocerebral trauma, general disturbances of concentration, concentration disturbances in children suffering from learning and memory problems, vascular dementia, dementia associated with Lewy bodies, dementia associated with degeneration of the frontal lobes including Pick's syndrome, Parkinson's disease, progressive nuclear palsy, dementia associated with corticobasal degeneration, amyolateral sclerosis (ALS), Huntington's disease, multiple sclerosis, thalamic degeneration, Creutzfeld-Jacob dementia, new variant Creutzfeld-Jacob dementia, HIV dementia, schizophrenia associated with dementia or Korsakoff's psychosis.

The compounds which are used in accordance with the invention can exist in stereoisomeric forms which either relate to each other as image and mirror image (enantiomers) or which do not relate to each other as image and mirror image (diastereomers). The invention relates to the use of both the enantiomers and the diastereomers or their respective mixtures. The racemic forms can, like the diastereomers, be separated, in a known manner, into the stereoisomerically hombgeneous. components.

The substances which are used in accordance with the invention can also be present as salts. Within the context of the invention, preference is given to using physiologically harmless salts.

Physiologically harmless salts can be salts of the compounds used in accordance with the invention with inorganic or organic acids. Preference is given to salts with inorganic acids such as hydrochloric acid, hydrobromic acid, phosphoric acid or sulfuric acid, or salts with organic carboxylic or sulfonic acids, such as acetic acid, maleic acid, fumaric acid, malic acid, citric acid, tartaric acid, lactic acid, benzoic acid, or methanesulfonic acid, ethanesulfonic acid, phenylsulfonic acid, toluenesulfonic acid or naphthalenedisulfonic acid.

Physiologically harmless salts can also be metal salts or ammonium salts of the compounds according to the invention. Particular preference is given, for example, to sodium, potassium, magnesium or calcium salts and to ammonium salts which are derived from ammonia or organic amines such as ethylamine, diethylamine, triethylamine, diethanolamine, triethanolamine, dicyclohexylamine, dimethylaminoethanol, arginine, lysine, ethylenediamine or 2-phenylethylamine.

(C₃-C₈)-Cycloalkyl is cyclopropyl, cyclopentyl, cyclobutyl, cyclohexyl, cycloheptyl or cyclooctyl. Those which may be mentioned as being preferred are: cyclopropyl, cyclopentyl and cyclohexyl.

(C₁-C₈) -Alkyl, (C₁-C₆) -alkyl or, respectively, (C₁-C₄)-alkyl is a straight-chain or branched alkyl radical having from 1 to 8, from 1 to 6 or, respectively, from 1 to 4 carbon atoms. Examples which may be mentioned are: methyl, ethyl, n-propyl, isopropyl, n-butyl, isobutyl, tert-butyl, n-pentyl and n-hexyl. A straight-chain or branched alkyl radical having from 1 to 4 carbon atoms is preferred. A straight-chain or branched alkyl radical having from 1 to 3 carbon atoms is particularly preferred.

(C₁-C₆)-Alkoxy is a straight-chain or branched alkoxy radical having from 1 to 6 carbon atoms. Examples which may be mentioned are: methoxy, ethoxy, n-propoxy, isopropoxy, n-butoxy, isobutoxy, tert-butoxy, n-pentoxy and n-hexoxy. A straight-chain or branched alkoxy radical having from 1 to 4 carbon atoms is preferred. A straight-chain or branched alkoxy radical having from 1 to 3 carbon atoms is particularly preferred.

Halogen is generally fluorine, chlorine, bromine and iodine. Fluorine, chlorine and bromine are preferred. Fluorine and chlorine are particularly preferred.

A 5- to 6-membered aromatic heterocycle having up to 3 hetero atoms from the series S, O and/or N is, for example, pyridyl, pyrimidyl, pyridazinyl, thienyl, furyl, pyrrolyl, thiazolyl, oxazolyl or imidazolyl. Pyridyl, pyrimidyl, pyridazinyl, furyl and thienyl are preferred.

Preference is given to the use, according to the invention, of compounds of the general formula (I)

in which

R¹ is methyl or ethyl,
R² is straight-chain or branched alkyl having up to 3 carbon atoms or is (C₃-C₆)-cycloalkyl,
R³ is straight-chain or branched alkyl having up to 3 carbon atoms,
R⁴ and R⁵ are identical or different and are hydrogen, (C₁-C₄)-alkoxy or hydroxyl or are (C₁-C₇)-alkyl which is optionally substituted, up to 3 times, identically or differently, by hydroxyl, (C₁-C₄)-alkoxy or radicals of the formulae 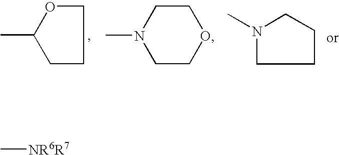
   in which
   R⁶ and R⁷ are identical or different and are hydrogen or methyl,
   and/or, for its part, (C₁-C₇)-alkyl is optionally substituted by phenyl or phenoxy which, for their part, are optionally substituted, once to three times, identically or differently, by fluorine, chlorine, hydroxyl, (C₁-C₄)-alkoxy or (C₁-C₄)-alkyl or by a radical of the formula —SO₂NH₂,
      or
R⁴ is hydrogen or methyl,
   and
R⁵ is radicals of the formulae 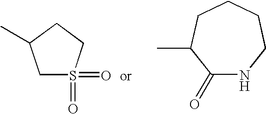
   or
   is phenyl which is optionally substituted, up to 3 times, identically or differently, by fluorine, chlorine, acetyl or (C₁-C₄)-alkoxy or by radicals of the formulae 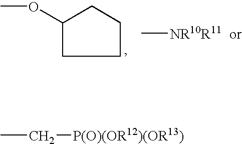
   in which
   R¹⁰ and R¹¹ are identical or different and are hydrogen or methyl,
   R¹² and R¹³ are identical or different and are hydrogen or methyl, or
   R⁴ and R⁵, together with the nitrogen atom to which they are bonded, are radicals of the formulae 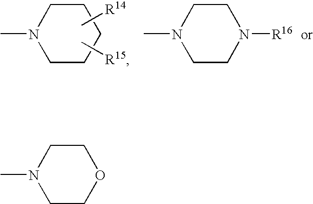
   in which
   R¹⁴ and R¹⁵ are identical or different and are hydroxyl, hydrogen or (C₁ -C₃)-alkyl which is optionally substituted by hydroxyl,
   or
   R¹⁴ is hydrogen
   and
   R¹⁵ is a radical of the formula 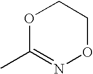
   or
   R¹⁴ and R¹⁵ together form a radical of the formula ═N—O—CH₃,
   R¹⁶ is hydrogen or (C₁-C₅)-alkyl which is optionally substituted by hydroxyl, or is pyridyl, pyrimidyl, furyl, pyrryl or thienyl, and the salts, hydrates, N-oxides and isomeric forms thereof.

Particular preference is given to the use, according to the invention, of compounds of the general formula (I) in which
R¹ is methyl or ethyl,
R² is n-propyl or cyclopentyl,
R³ is methyl, ethyl or n-propyl,
R⁴ and R⁵ are identical or different and are hydrogen, (C₁-C₃)-alkoxy or hydroxyl or are (C₁-C₆)-alkyl which is optionally substituted, up to 3 times, identically or differently, by hydroxyl or (C₁-C₃)-alkoxy or by radicals of the formulae 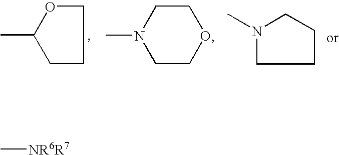
   in which
   R⁶ and R⁷ are identical or different and are hydrogen or methyl,
   and/or, for its part, (C₁-C₆)-alkyl is optionally substituted by phenyl or phenoxy which, for their part, are optionally substituted, once to three times, identically or differently, by fluorine, hydroxyl or methoxy or by a radical of the formula —SO₂NH₂,
      or
R⁴ is hydrogen or methyl
   and
R⁵ is radicals of the formulae 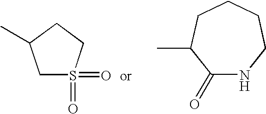
   or
   is phenyl which is optionally substituted, up to 3 times, identically or differently, by fluorine, acetyl or methoxy or by radicals of the formulae 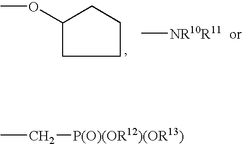
   in which
   R¹⁰ and R¹¹ are identical or different and are hydrogen or methyl,
   R¹² and R¹³ are methyl,
      or
R⁴ and R⁵, together with the nitrogen atom to which they are bonded, are radicals of the formulae 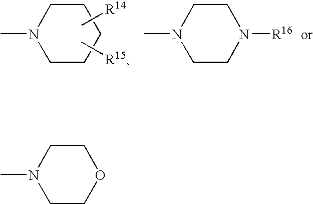
   in which
   R¹⁴ and R¹⁵ are identical or different and are hydroxyl or hydrogen or a radical of the formula —(CH₂)₂—OH,
   or
   R¹⁴ is hydrogen
   and
   R¹⁵ is a radical of the formula 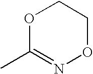
   or
   R¹⁴ and R¹⁵ together form a radical of the formula ═N—O—CH₃,
   R¹⁶ is hydrogen, pyrimidyl or a radical of the formula —(CH₂) ₂—OH
      and the salts, hydrates, hydrates of the salts, N-oxides and isomeric forms thereof.

Very particular preference is given to the use according to the invention of the following compounds: 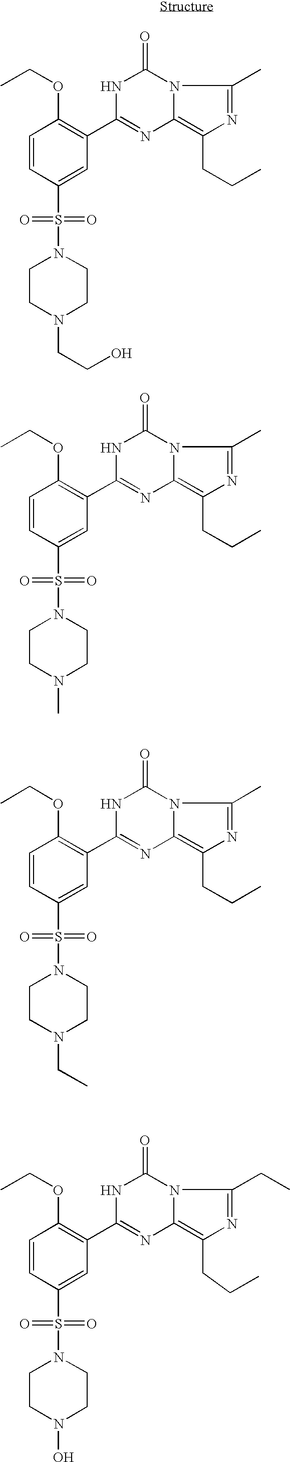 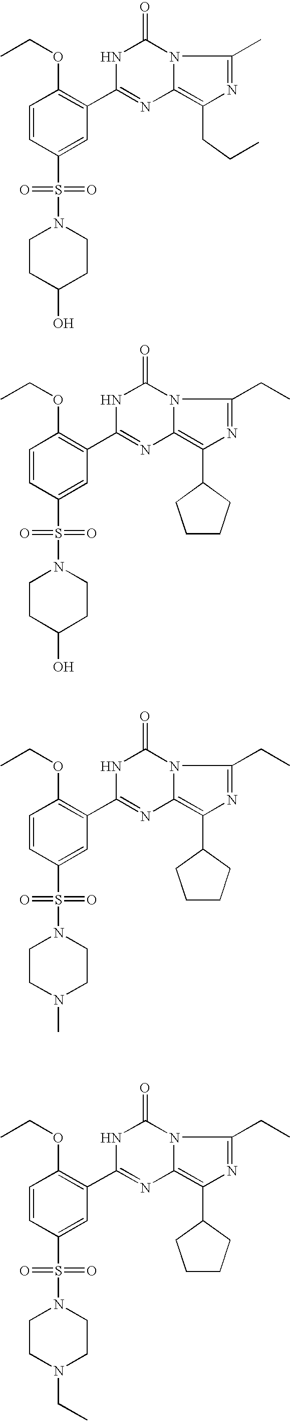 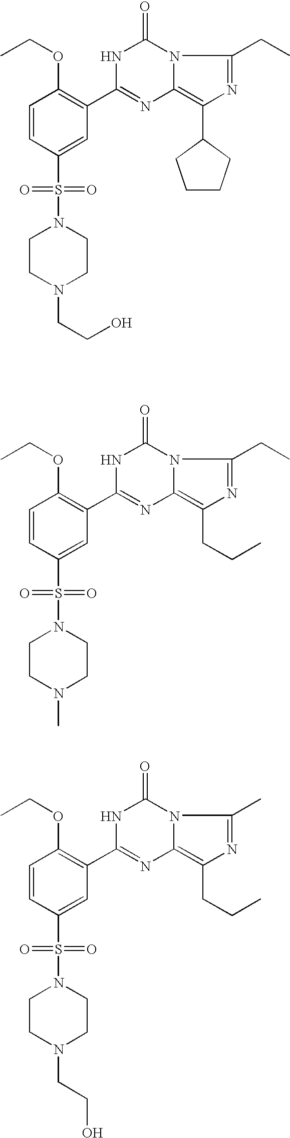 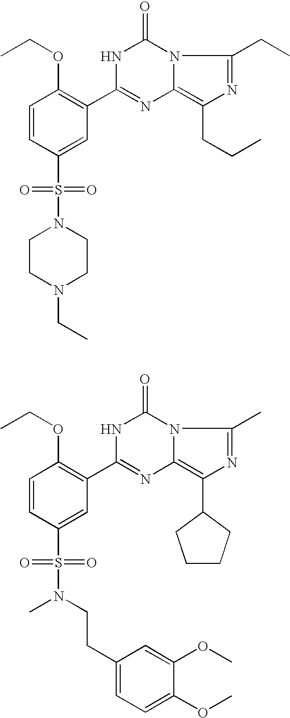
and the salts, hydrates, hydrates of the salts, N-oxides and isomeric forms thereof.

The compounds which are used according to the invention, and their preparation, are described in WO/0147928. The disclosure of WO/0147928 is expressly incorporated herein by reference.

The compounds which are used in accordance with the invention are suitable for the prophylaxis and/or treatment of diseases in which an increase in the concentration of cGMP is curative, i.e. diseases which are connected to cGMP-regulated processes (usually referred to in English simply as ‘cGMP-related diseases’).

The relaxing effect on smooth musculature makes them suitable for treating diseases in which an improvement and/or cure of a syndrome can be achieved by improving the microcirculation of a tissue which contains a cGMP-metabolizing phosphodiesterase.

In this connection, the inhibition of one or more phosphodiesterases leads to the CGMP concentration being increased. As a result, the compounds are of interest for all therapies in which an increase in the concentration of cGMP can be assumed to be curative.

In particular, the abovementioned compounds are for producing a pharmaceutical for the treatment and/or prophylaxis of coronary heart disease, cardiac insufficiency, pulmonary hypertension, bladder diseases, prostate hyperplasia, nitrate-induced tolerance and diseases of the eye such as glaucoma, for the treatment or prophylaxis of central retinal or posterior cilliary arterial occlusion, central retinal venous occlusion, optical neuropathy such as anterior ischemic optical neuropathy and glaucomatous optical neuropathy, and also of macular degeneration and diabetes, in particular of diabetic gastroparesis, and for the treatment of disturbances of peristalsis of the stomach and esophagus, of female infertility, premature labor, preeclampsia, alopecia, psoriasis, the renal syndrome, cystic fibrosis and cancer.

In particular, the abovementioned compounds are also used for producing pharmaceuticals for improving perception, for improving concentration performance, for improving learning performance and/or memory performance, in particular when the disturbance is a consequence of dementia, for improving perception, concentration performance and learning performance and/or memory performance after cognitive disturbances, as occur, in particular, in situations/diseases/syndromes such as mild cognitive impairment, age-associated learning and memory disturbances, age-associated memory loss, vascular dementia, craniocerebral trauma, stroke, dementia which occurs after strokes (post-stroke dementia), posttraumatic craniocerebral trauma, general disturbances of concentration, disturbances of concentration in children suffering from learning and memory problems, vascular dementia, dementia associated with Lewy bodies, dementia associated with degeneration of the frontal lobes including Pick's syndrome, Parkinson's disease, progressive nuclear palsy, dementia associated with corticobasal degeneration, amyolateralsclerosis (ALS), Huntington's disease, multiple sclerosis, thalamic degeneration, Creutzfeld-Jacob dementia, new variant Creutzfeld-Jacob dementia, HIV dementia, schizophrenia associated with dementia or Korsakoff's psychosis.

The activity of the phosphordiesterases (PDES) can be determined as follows. The cGMP-stimulatable PDE II, the cGMP-inhibitable PDE III and the cAMP-specific PDE IV were isolated either from pig heart myocardium or beef heart myocardium. The Ca²⁺-calmodulin-stimulatable PDE I was isolated from pig aorta, pig brain or, preferably, bovine aorta. The cGMP-specific PDE V was obtained from pig small intestine, pig aorta, human blood platelets and, preferably, bovine aorta. Purification was effected by means of anion exchange QR chromatography on Pharmacia MonoQ^{R}, essentially in accordance with the method of M. Hoey and Miles D. Houslay, Biochemical Pharmacology, Vol. 40, 193-202 (1990) and C. Lugman et al. Biochemical Pharmacology Vol. 35 1743-1751 (1986).

The enzyme activity is determined in a 100 μl test mixture, in 20 mM tris/HCl buffer, pH 7.5 which contains 5 MM MgCl₂, 0.1 mg of bovine serum albumin/ml and 800 Bq of either ³HcAMP or ³HcGMP. The final concentration of the appropriate nucleotides is 10⁻⁶ mol/l. The reaction is started by adding the enzyme; the quantity of enzyme is calculated such that approx. 50% of the substrate is converted during the incubation time of 30 min. In order to test the cGMP-stimulatable PDE II, HcAMP is used as the substrate and 10⁻⁶ mol of unlabeled cGMP/1 is added to the mixture. In order to test the Ca²⁺-calmodulin-dependent PDE I, 1 μM CaCl₂ and 0.1 μM calmodulin are also added to the reaction mixture. The reaction is stopped by adding 100 μl of acetonitrile which contains 1 mM cAMP and 1 mM AMP. 100 μl of the reaction mixture are fractionated by HPLC and the cleavage products are determined quantitatively on line using a flow-through scintillation counter. The substance concentration at which the reaction rate is reduced by 50% is measured. In addition, the “phosphodiesterase [³H] cAMP-SPA enzyme assay” and the “phosphodiesterase [³H] cGMP-SPA enzyme assay” from Amersham Life Science were used for the testing. The test was carried out in accordance with the experimental protocol specified by the manufacturer. The [³H] cAMP SPA assay was used for determining the activity of PDE II, with 10⁻⁶ M cGMP being added to the reaction mixture in order to activate the enzyme. For measuring PDE I, 10⁻⁷ M calmodulin and 1 μm CaCl₂ were added to the reaction mixture. PDE V was measured using the [³H] cGMP SPA assay.

Memory performance can be determined by means of an object recognition test. This test is used to measure the ability of rats (and mice) to distinguish between known and unknown objects.

test is carried out as described in Blokland et al., NeuroReport 1998, 9, 4205; Ennaceur et al., Behav. Brain Res. 1988, 31, 47-59; Ennaceur et al., Psychopharmacology 1992, 109, 321-330; Prickaerts et al., Eur. J. Pharmacol. 1997, 337, 125-136.

The inhibition of one or more phosphodiesterases of this type leads to an increase in the concentration of cGMP.

The compounds which are used in accordance with the invention, and their physiologically harmless salts (e.g. hydrochlorides, maleates or lactates) and hydrates, can be converted, in a known manner, into the customary formulations such as tablets, sugar-coated tablets, pills, granules, aerosols, syrups, emulsions, suspensions and solutions using inert, nontoxic, pharmaceutically suitable carrier substances or solvents. In this connection, the therapeutically active compound should in each case be present at a concentration of from about 0.5 to 90% by weight of the total mixture, i.e. in quantities which are sufficient to achieve the specified dosage latitude.

The formulations are prepared, for example, by extending the active compounds with solvents and/or carrier substances, where appropriate using emulsifiers and/or dispersing agents, with it being possible, for example when using water as diluent, to employ organic solvents as auxiliary solvents, where appropriate.

Administration is effected in a customary manner, preferably orally, transdermally or parenterally, for example by the perlingual, sublingual, conjunctival, otic, buccal, intravenous, nasal, rectal or inhalative route, or as an implant.

For use in humans, doses of from 0.001 to 50 mg/kg, preferably 0.01 mg/kg-20 mg/kg, are generally administered in the case of oral administration. In the case of parenteral administration, for example by way of mucus membranes by the nasal, buccal or inhalative route, a dose of 0.001 mg/kg-0.5 mg/kg is appropriate.

Despite this, it may be necessary, where appropriate, to depart from the abovementioned quantities depending on the body weight or the nature of the administration route, on the individual response to the medicament, on the nature of its formulation and on the time or interval at which the administration takes place. Thus, it may be sufficient, in some cases, to make do with less than the abovementioned minimum quantity while it is necessary to exceed the abovementioned upper limit in other cases. When relatively large quantities are being administered, it may be advisable to divide these into several single doses which are administered over the course of the day.

The compounds which are used in accordance with the invention are also suitable for being employed in veterinary medicine. For applications in veterinary medicine, the compounds, or their nontoxic salts, can be administered in a suitable formulation in conformity with the general practices of veterinary medicine. The veterinarian can specify the nature of the application and the dose in dependence on the nature of the animal to be treated.

1. A method for the treatment and/or prophylaxis of diseases in which an improvement in and/or a cure of a syndrome can be achieved by improving the microcirculation of a tissue which contains a cGMP-metabolizing phosphodiesterase comprising administering to a subject an effective amount of a cGMP-stimulating compound.

2. The method of claim 1, wherein said disease is selected from coronary heart disease, cardiac insufficiency, pulmonary hypertension, bladder diseases, prostate hyperplasia, nitrate-induced tolerance or diseases of the eye, central retinal or posterior cilliary arterial occlusion, central retinal venous occlusion, optical neuropathy, macular degeneration, diabetes, disturbances in the peristalsis of the stomach and esophagus, female infertility, premature labor, preeclampsia, alopecia, psoriasis, the renal syndrome, cystic fibrosis and cancer.

3. The method of claim 1 for improving perception, concentration performance and learning performance and/or memory performance, for improving perception, concentration performance, learning performance and/or memory performance following cognitive disturbances, age-associated learning and memory disturbances, age-associated memory loss, vascular dementia, craniocerebral trauma, stroke, dementia which occurs following strokes (post-stroke dementia), post-traumatic craniocerebral trauma, general disturbances of concentration, concentration disturbances in children suffering from learning and memory problems, vascular dementia, dementia associated with Lewy bodies, dementia associated with degeneration of the frontal lobes including Pick's syndrome, Parkinson's disease, progressive nuclear palsy, dementia associated with corticobasal degeneration, amyolateral sclerosis (ALS), Huntington's disease, multiple sclerosis, thalamic degeneration, Creutzfeld-Jacob dementia, new variant Creutzfeld-Jacob dementia, HIV dementia, schizophrenia associated with dementia or Korsakoff's psychosis.

4. The method of claim 1, wherein the cGMP-stimulating compound is selected from the imidazo[1 ,3,5 ]triazinone of the general formula (I) 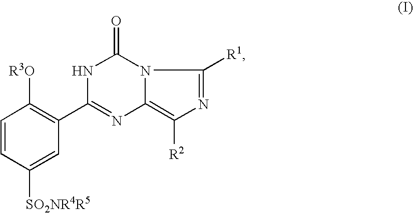
in which
R¹ is straight-chain or branched alkyl having up to 4 carbon atoms,
R² is straight-chain or branched alkyl having up to 4 carbon atoms or is (C₃-C₈)-cycloalkyl,
R³ is hydrogen or straight-chain or branched alkyl having up to 4 carbon atoms,
R⁴ and R⁵ are identical or different and are hydrogen, (C₁-C₆)-alkoxy or hydroxyl or are (C₁-C₈)-alkyl which is optionally substituted, up to 3 times, identically or differently, by hydroxyl, (C₁-C₆)-alkoxy or radicals of the formulae 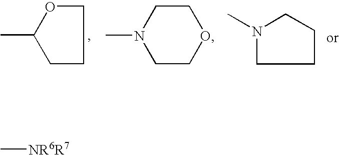
in which
R⁶ and R⁷ are identical or different and are hydrogen or (C₁-C₆)-alkyl,
and/or, for its part, (C₁-C₈)-alkyl is optionally substituted by phenyl or phenoxy which, for their part, are optionally substituted, once to three times, identically or differently, by halogen, hydroxyl, (C₁-C₆)-alkoxy, (C₁-C₆)-alkyl or a radical of the formula —SO₂NR⁸R⁹,
in which
R⁸ and R⁹ are identical or different and are hydrogen or (C1-C₆)-alkyl,
or
R⁴ is hydrogen or methyl
and
R⁵ is radicals of the formulae 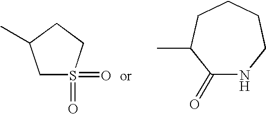
or
is phenyl which is optionally substituted, up to 3 times, identically or differently, by halogen, acetyl, (C₁-C₆)-alkoxy or radicals of the formulae 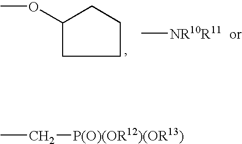
in which
R¹⁰ and R¹¹ are identical or different and are hydrogen or (C₁-C₄)-alkyl,
R¹² and R¹³ are identical or different and are hydrogen or (C₁-C₆)-alkyl,
or
R⁴ and R⁵, together with the nitrogen atom to which they are bonded, are radicals of the formulae 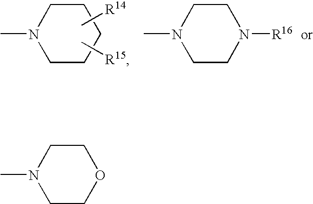
in which
R¹⁴ and R¹⁵ are identical or different and are hydroxyl, hydrogen or (C₁-C₄)-alkyl which is optionally substituted by hydroxyl,
or
R¹⁴ is hydrogen
and
R¹⁵ is a radical of the formula 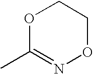
or
R¹⁴ and R¹⁵ together form a radical of the formula ═N—O—CH₃,
R¹⁶ is hydrogen or (C₁-C₆)-alkyl which is optionally substituted by hydroxyl, or
is a 5- to 6-membered, aromatic heterocycle having up to 3 hetero atoms from the series, S, N and/or O,
and the salts, hydrates, hydrates of the salts, N-oxides and isomeric forms thereof.

5. The method of claim 4, wherein the compound is selected from the compounds of the general formula (I)
in which
R¹ is methyl or ethyl,
R² is straight-chain or branched alkyl having up to 3 carbon atoms or is (C₃-C₆)-cycloalkyl,
R³ is straight-chain or branched alkyl having up to 3 carbon atoms,
R⁴ and R⁵ are identical or different and are hydrogen, (C₁-C₄)-alkoxy or hydroxyl or are (C₁-C₇)-alkyl which is optionally substituted, up to 3 times, identically or differently, by hydroxyl, (C₁-C₄)-alkoxy or radicals of the formulae 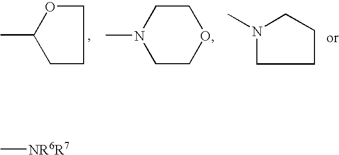
in which
R⁶ and R⁷ are identical or different and are hydrogen or methyl, and/or, for its part, (C₁-C₇)-alkyl is optionally substituted by phenyl or phenoxy which, for their part, are optionally substituted, once to three times, identically or differently, by fluorine, chlorine, hydroxyl, (C₁-C₄)-alkoxy or (C₁-C₄)-alkyl or by a radical of the formula —SO₂NH₂, or
R⁴ is hydrogen or methyl,
and
R⁵ is radicals of the formulae 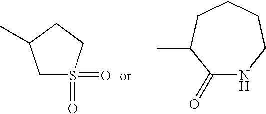
or
is phenyl which is optionally substituted, up to 3 times, identically or differently, by fluorine, chlorine, acetyl or (C₁-C₄)-alkoxy or by radicals of the formulae 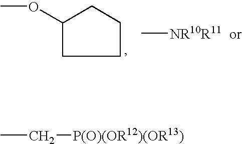
in which
R¹⁰ and R¹¹ are identical or different and are hydrogen or methyl,
R¹² and R¹³ are identical or different and are hydrogen or methyl,
or
R⁴ and R⁵, together with the nitrogen atom to which they are bonded, are radicals of the formulae 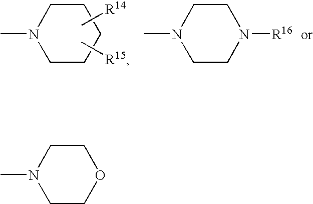
in which
R¹⁴ and R¹⁵ are identical or different and are hydroxyl, hydrogen or (C₁-C₃)-alkyl which is optionally substituted by hydroxyl,
or
R¹⁴ is hydrogen
and
R¹⁵ is a radical of the formula 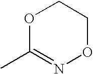
or
R¹⁴ and R¹⁵ together form a radical of the formula ═N—O—CH₃,
R¹⁶ is hydrogen or (C₁-C₅)-alkyl which is optionally substituted by hydroxyl, or is pyridyl, pyrimidyl, furyl, pyrryl or thienyl,
and the salts, hydrates, hydrates of the salts, N-oxides and isomeric forms thereof.

6. The method of claim 4, wherein the compound is selected from the compound of the general formula (I)
in which
R¹ is methyl or ethyl,
R² is n-propyl or cyclopentyl,
R³ is methyl, ethyl or n-propyl,
R⁴ and R⁵ are identical or different and are hydrogen, (C₁-C₃)-alkoxy or hydroxyl or are (C₁-C₆)-alkyl which is optionally substituted, up to 3 times, identically or differently, by hydroxyl or (C₁-C₃)-alkoxy or by radicals of the formulae 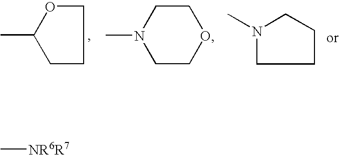
in which
R⁶ and R⁷ are identical or different and are hydrogen or methyl, and/or, for its part, (C₁-C₆)-alkyl is optionally substituted by phenyl or phenoxy which, for their part, are optionally substituted, once to three times, identically or differently, by fluorine, hydroxyl or methoxy or by a radical of the formula —SO₂NH₂,
or
R⁴ is hydrogen or methyl
and
R⁵ is radicals of the formulae 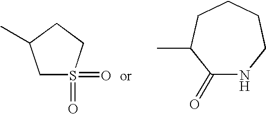
or
is phenyl which is optionally substituted, up to 3 times, identically or differently, by fluorine, acetyl or methoxy or by radicals of the formulae 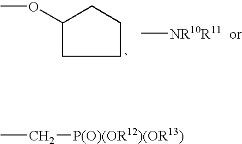
in which
R¹⁰ and R¹¹ are identical or different and are hydrogen or methyl,
R¹² and R¹³ are methyl,
or
R⁴ and R⁵, together with the nitrogen atom to which they are bonded, are radicals of the formulae 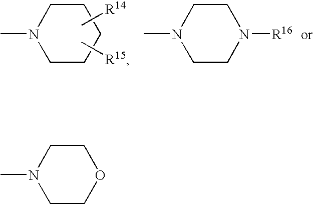
in which
R¹⁴ and R¹⁵ are identical or different and are hydroxyl or hydrogen or a radical of the formula —(CH₂)₂—OH,
or
R¹⁴ is hydrogen
and
R¹⁵ is a radical of the formula 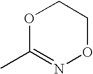
or
R¹⁴ and R¹⁵ together form a radical of the formula ═N—O—CH₃,
R¹⁶ is hydrogen, pyrimidyl or a radical of the formula —(CH₂)₂—OH
and the salts, hydrates, hydrates of the salts, N-oxides and isomeric forms thereof.

7. A pharmaceutical for the treatment and/or prophylaxis of diseases in which an improvement in and/or a cure of a syndrome can be achieved by improving the microcirculation of a tissue which contains a cGMP-metabolizing phosphodiesterase, which pharmaceutical comprises at least one cGMP-stimulating compound.

8. A pharmaceutical for the treatment and/or prophylaxis of coronary heart disease, cardiac insufficiency, pulmonary hypertension, bladder diseases, prostate hyperplasia, nitrate-induced tolerance or diseases of the eye, central retinal or posterior cilliary arterial occlusion, central retinal venous occlusion, optical neuropathy, macular degeneration, diabetes, disturbances of the peristalsis of the stomach and esophagus, female infertility, premature labor, preeclampsia, alopecia, psoriasis, renal syndrome, cystic fibrosis and/or cancer, which pharmaceutical comprises at least one cGMP-stimulating compound.

9. A pharmaceutical for improving perception, concentration performance, learning performance and/or memory performance, for improving perception, concentration performance, learning performance and/or memory performance following cognitive disturbances, age-associated learning and memory disturbances, age-associated memory loss, vascular dementia, craniocerebral trauma, stroke, dementia which occurs after strokes (post-stroke dementia), post-traumatic craniocerebral trauma, general disturbances of concentration, concentration disturbances in children suffering from learning and memory problems, vascular dementia, dementia associated with Lewy bodies, dementia associated with degeneration of the frontal lobes including Pick's syndrome, Parkinson's disease, progressive nuclear palsy, dementia associated with corticobasal degeneration, amyolateralsclerosis (ALS), Huntington's disease, multiple sclerosis, thalamic degeneration, Creutzfeld-Jacob dementia, new variant Creutzfeld-Jacob dementia, HIV dementia, schizophrenia associated with dementia or Korsakoff's psychosis, which pharmaceutical comprises at least one cGMP-stimulating compound.

10. A pharmaceutical which comprises at least one compound imidazo[1,3,5]triazinone of the general formula (I) 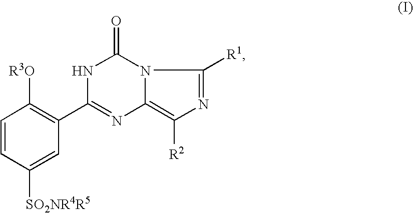
in which
R¹ is straight-chain or branched alkyl having up to 4 carbon atoms,
R² is straight-chain or branched alkyl having up to 4 carbon atoms or is (C₁-C₈)-cycloalkyl,
R³ is hydrogen or straight-chain or branched alkyl having up to 4 carbon atoms,
R⁴ and R⁵ are identical or different and are hydrogen. (C₁-C₆)-alkoxy or hydroxyl or are (C₁-C₈)-alkyl which is optionally substituted, up to 3 times, identically or differently, by hydroxyl, (C₁-C₆)-alkoxy or radicals of the formulae 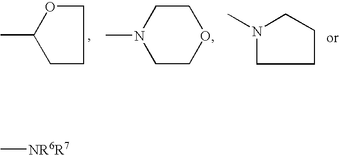
in which
R⁶ and R⁷ are identical or different and are hydrogen or (C₁-C₆)-alkyl,
and/or for its part, (C₁-C₈)-alkyl is optionally substituted by phenyl or phenoxy which, for their part, are optionally substituted, once to three times, identically or differently, by halogen, hydroxyl, (C₁-C₆)-alkoxy, (C₁-C₆)-alkyl or a radical of the formula —SO₂NR⁸R⁹,
in which
R⁸ and R⁹ are identical or different and are hydrogen or (C₁-C₆)-alkyl,
or
R⁴ is hydrogen or methyl
and
R⁵ is radicals of the formulae 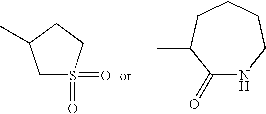
or
is phenyl which is optionally substituted, up to 3 times, identically or differently, by halogen, acetyl, (C₁-₆)-alkoxy or radicals of the formulae 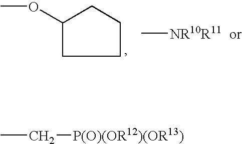
in which
R¹⁰ and R¹¹ are identical or different and are hydrogen or (C ₁-C₄)-alkyl
R¹² and R¹³ are identical or different and are hydrogen or (C₁-C₆)-alkyl,
or
R⁴ and R⁵, together with the nitrogen atom to which they are bonded, are radicals of the formulae 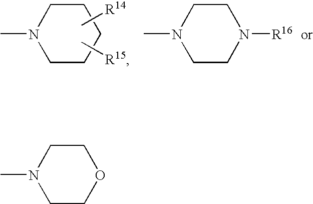
in which
R¹⁴ and R¹⁵ are identical or different and are hydroxyl, hydrogen or (C₁-C₄-alkyl which is optionally substituted by hydroxyl,
or
R¹⁴ is hydrogen
and
R¹⁵ is a radical of the formula 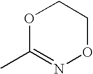
or
R¹⁴ and R¹⁵ together form a radical of the formula ═N—O—CH₃,
R¹⁶ is hydrogen or (C₁-C₅)-alkyl which is optionally substituted by hydroxyl, or
is a 5- to 6-membered aromatic heterocycle having up to 3 hetero atoms from the series, S, N and/or O,
and the salts, hydrates, hydrates of the salts, N-oxides and isomeric forms thereof.
